SEQUENCE LISTING (3 sequences)

SEQ ID NO: 1 - DNA, 4236 bp, Homo sapiens
ggtgggcggggggaggaggcggccctagcgccattttgtgggagcgaagcggtggctgggctgcgcttgggtccgtcgctgcttcggtgtccctgtcgggcttcccagcagcggcctagcgggaaaagtaaaagatgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtagacagtggggttgtggttggttggtatagaatggtgggaattcaaatttttctaaactcatggtaagtatattgtaaaatacatatgtactaagaattttcaaaattggtttgttcagtgtggagtatattcagcagtatttttgacatttttctttagaaaaaggaagagctaaaggaattttataagttttgttacatgaaaggttgaaatattgagtggttgaaagtgaactgctgtttgcctgattggtaaaccaacacactacaattgatatcaaaaggtttctcctgtaatattttatccctggacttgtcaagtgaattctttgcatgttcaaaacggaaaccattgattagaactacattctttaccccttgttttaatttgaaccccaccatatggatttttttccttaagaaaatctccttttaggagatcatggtgtcacagtgtttggttcttttgttttgttttttaacacttgtctcccctcatacacaaaagtacaatatgaagccttcatttaatctctgcagttcatctcatttcaaatgtttatggaagaagcacttcattgaaagtagtgctgtaaatattctgccataggaatactgtctacatgctttctcattcaagaattcgtcatcacgcatcacaggccgcgtctttgacggtgggtgtcccatttttatccgctactctttatttcatggagtcgtatcaacgctatgaacgcaaggctgtgatatggaaccagaaggctgtctgaacttttgaaaccttgtgtgggattgatggtggtgccgaggcatgaaaggctagtatgagcgagaaaaggagagagcgcgtgcagagacttggtggtgcataatggatattttttaacttggcgagatgtgtctctcaatcctgtggctttggtgagagagtgtgcagagagcaatgatagcaaataatgtacgaatgttttttgcattcaaaggacatccacatctgttggaagacttttaagtgagtttttgttcttagataacccacattagatgaatgtgttaagtgaaatgatacttgtactccccctacccctttgtcaactgctgtgaatgctgtatggtgtgtgttctcttctgttactgatatgtaagtgtggcaatgtgaactgaagctgatgggctgagaacatggactgagcttgtggtgtgctttgcaggaggacttgaagcagagttcaccagtgagctcaggtgtctcaaagaagggtggaagttctaatgtctgttagctacccataagaatgctgtttgctgcagttctgtgtcctgtgcttggatgctttttataagagttgtcattgttggaaattcttaaataaaactgatttaaataatatgtgtctttgttttgcagccctgaatgcaaagaattcatagcagttaattccccttttttgacccttttgagatggaactttcataaagtttcttggcagtagtttattttgcttcaaataaacttatttgaaaagttgtctcaagtcaaatggattcatcacctgtcatgcattgacacctgatacccagacttaattggtatttgttcttgcattggccaaagtgaaaatttttttttttcttttgaaatctagttttgaataagtctgggtgaccgcacctaaaatggtaagcagtaccctccggctttttcttagtgcctctgtgcatttgggtgatgttctatttacatggcctgtgtaaatctccattgggaagtcatgccttctaaaaagattcttatttgggggagtgggcaaaatgttgattattttctaatgctttgtagcaaagcatatcaattgaaaagggaatatcagcaccttcctagtttgggatttgaaaagtggaattaattgcagtagggataaagtagaagaaaccacaaattatcttgtgcctgaaatccattaagaggcctgatagctttaagaattagggtgggttgtctgtctggaagtgttaagtggaatgggctttgtcctccaggaggtgggggaatgtggtaacattgaatacagttgaataaaatcgcttacaaaactcacactctcacaatgcattgttaagtatgtaaaagcaataacattgattctctgttgtacttttttgtaactaattctgtgagagttgagctcattttctagttggaagaatgtgatatttgttgtgttggtagtttacctaatgcccttacctaattagattatgataaataggtttgtcattttgcaagttacataaacatttatcaatgaagtcatcctttagacttgtaatcgccacattgtttcattattcagtttcctctgtaaagggatcttgagttgttttaattttttttttctgcatctgaatctgcatgatttccaaaccctgtaccatctgaattttgcattttagcacttgcactattactcagcagcagtaacatggtaacacttaaaatggtactcggggacctccaaagactaaactgacaagccttcaaggagcccaggggtaagttaacttgtcaacggcatggtttaatcccttctttacacttgtgtaaatttcagttactggtcatagaaggctttcaatgttgagtggccttttattaacatgtttatggtactgcatagatacgggtatttattttaccctaagaagattttgaagtttaaaagtacttaaactatttggcaaagatttgtttttaaaaatctatttggtcaatctaaatgcattcattctaaaaaattttttgaaccagataaataaaatttttttttgacaccacaaaaaaaaaaaaaaaaaaaa

SEQ ID NO: 2 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 3 - protein, 11 aa, Homo sapiens
CMDSKSSGWGM

(19) United States
(12) Patent Application Publication
(10) Pub. No.: US 2015/0111777 A1
(43) Pub. Date: Apr. 23, 2015

(54) BIOMARKER FOR EARLY NEURODEGENERATION DETECTION

(71) Applicant: Kansas City University of Medicine and Biosciences, Kansas City, MO (US)

(72) Inventor: Abdulbaki Agbas, Kansas City, MO (US)

(21) Appl. No.: 14/495,071

(22) Filed: Sep. 24, 2014

Related U.S. Application Data
(60) Provisional application No. 61/881,683, filed on Sep. 24, 2013.

(51) Int. Cl. G01N 33/68

(52) U.S. Cl. CPC G01N 33/6896; G01N 2333/4704; G01N 2800/2821; G01N 2800/52; G01N 2458/00
USPC 506/9; 436/501; 435/7.92; 435/7.1; 530/387.9; 530/391.3; 435/183

(57) ABSTRACT

The present invention relates to compositions, methods, and kits that can be used to assess neurodegeneration associated diseases and conditions in a subject by detecting the level of TDP-43 protein in platelets. The compositions, methods, and kits are suitable for neurodegeneration associated disease diagnosis, prognosis and treatment evaluation.

### FIELD OF THE INVENTION

The present invention generally relates to compositions, methods and kits useful for diagnostic, prognostic and therapeutic evaluation of neurodegenerative diseases or conditions by detecting the level of TDP-43 protein in platelets of a subject.

### BACKGROUND OF THE INVENTION

Neurodegeneration, a process in which nerve cells in the central or peripheral nervous system gradually lose function and ultimately die, afflicts millions of people worldwide. The risk of being affected by a neurodegenerative disease increases dramatically with age. Alzheimer's disease and Parkinson's disease are the most frequent types of neurodegenerative disease. Additional neurodegenerative diseases and conditions include corticobasal degeneration, Lewy-body dementia, frontotemporal lobar degeneration, amyotrophic lateral sclerosis, and nerve degeneration, as well as others. Many of these conditions are slow progressing and progress for decades before symptoms arise.

There is a great need to improve early detection and move diagnosis earlier in the course of neurodegeneration. The early diagnosis of neurodegenerative diseases is critical for starting early, disease-specific treatment. Current early diagnostic tests rely on brain imaging technology or require cerebrospinal fluid samples; however, none show high reliability and sensitivity. Therefore, identifying biomarkers, especially protein biomarkers, is an urgent task. More specifically, new biomarkers that are sensitive, specific, reliable, reproducible, easily obtainable, non-invasive, and affordable are in great demand for neurodegenerative diagnosis and treatment.

Aggregates of TAR DNA-binding protein 43 (TDP-43, transactive response DNA binding protein 43 kDa, UniProtKB/Swiss-Prot No. Q13148) have been found in degenerating cells during neurodegeneration. TDP-43 is a RNA binding protein containing two RNA-recognition motifs (RRM), a nuclear localization signal (NLS) and a nuclear export signal (NES). The protein is normally concentrated in the nucleus, but also shuttles back and forth between the nucleus and cytoplasm. TDP-43 is a global 49473611.1 regulator of gene expression and is involved in the regulation of transcription and multiple aspects of RNA processing, including splicing, stability, transport, translation and microRNA maturation. TDP-43 interacts with many proteins and RNAs and functions in multi-protein/RNA complexes. Elevated levels of the TDP-43 protein have been identified in individuals diagnosed with chronic traumatic encephalopathy, a condition that often mimics ALS and that has been associated with athletes who have experienced multiple concussions and other types of head injury. TDP-43 is also a neuronal activity response factor in the dendrites of hippocampal neurons, with roles in regulating mRNA stability, transport and local translation in neurons. Mutations in the TARDBP gene are associated with neurodegenerative disorders including frontotemporal lobar degeneration and amyotrophic lateral sclerosis (ALS). While TDP-43 has been associated with neurodegeneration, methods of detecting TDP-43 levels require invasive measures to collect spinal fluid or nerve tissue.

Accordingly, a need still exists for sensitive and reliable detection methods that rely on less invasive sample collection and provide early detection of neurodegeneration. Compositions and methods exploiting TDP-43 based detection methods are needed to further medical research and provide improved diagnostic, prognostic, and therapeutic resources. The present invention provides compositions and methods for detecting TDP-43 levels in platelets, offering early neurodegeneration detection.

### SUMMARY OF INVENTION

The present disclosure relates to the discovery that TDP-43 protein, which aggregates in degenerating cells during neurodegenerative conditions or diseases, exists also in platelets and TDP-43 platelet levels correspond to disease conditions. Thus, by analyzing levels of TDP-43 protein in platelets, it is possible to detect and monitor neurodegenerative conditions associated with TDP-43 aggregation. By monitoring for changes in TDP-43 levels in platelets, the presence of neurodegenerative diseases or conditions can be predicted. Accordingly, the present disclosure provides a method for the detection of neurodegenerative conditions or diseases in a subject. Preferably, the neurodegenerative condition or disease is detected early.

The present disclosure provides methods for neurodegenerative disease detection, diagnosis, and monitoring by using platelet TDP-43 level as a biomarker. In some aspects, the method includes detecting or measuring the post-translational modification status of TDP-43 in platelets to evaluate neurodegeneration associated TDP-43 aggregation. In some aspects, the present invention includes methods of monitoring neurodegeneration progression by assessing the level of the TDP-43 protein in platelets over a period of time. In some aspects, the TDP-43 protein is post-transcriptionally modified. Such post-transcriptional modifications include, without limitation, phosphorylation, glycosylation, ubiquitination, hyper phosphorylation, truncation, SUMOylation, other post-transcriptional modifications known in the art, and combinations thereof.

In some aspects, the TDP-43 protein is phosphorylated. In some aspects, the TDP-43 protein is hyper phosphorylated. The TDP-43 protein may be phosphorylated at any amino acid residue capable of being phosphorylated, such as at one or more phosphomonoesters, phosphoramidates, acylphosphates, thiophosphates, or combinations thereof. Suitable phophoramino acids include serines, threonines, tyrosines, histidines, arginines, lysines, aspartates, glutamates, cysteines, and combinations thereof. In some aspects, the TDP-43 protein is phosphorylated at one or more serine residues. In some aspects, the TDP-43 protein is phosphorylated at one or more threonine residues. In some aspects, the TDP-43 protein is phosphorylated at one or more tyrosine residues. In some aspects, the TDP-43 protein is phosphorylated at one or more histidine residues. In some aspects, the TDP-43 protein is phosphorylated at one or more arginine residues. In some aspects, the TDP-43 protein is phosphorylated at one or more lysine residues. In some aspects, the TDP-43 protein is phosphorylated at one or more aspartate residues. In some aspects, the TDP-43 protein is phosphorylated at one or more glutamate residues. In some aspects, the TDP-43 protein is phosphorylated at one or more cysteine residues.

Some aspects of the invention provide methods of evaluating neurodegenerative disease (ND) in a subject. Some aspects of the methods include obtaining a sample comprising platelets from the subject; adding a binding agent of TDP-43 protein to the sample; subjecting the sample to conditions allowing the formation of the binding complexes between the binding agent and the TDP-43 protein; and, measuring the amount of the binding complexes and comparing that with a control sample. In some aspects an increased amount of the binding complexes in the sample in comparison to that in the control sample from a non-ND subject is indicative of the presence of ND in the subject.

In some aspects, platelets are isolated from a subject at a first time point. The platelets are analyzed for the level of TDP-43 protein. At a second, later time point, platelets are isolated from the subject and analyzed for the level of TDP-43. Next, the level of TDP-43 from the platelets of the first sample are compared to the level of TDP-43 from the platelets of the second sample. An increase in the level of TDP-43 in the platelets from the second sample, compared to the level of TDP-43 in the platelets from the first sample is indicative of a neurodegenerative disease or condition. Alternatively, a decrease or no change in the level of TDP-43 in the platelets from the second sample, compared to the level of TDP-43 in the first sample is indicative of a lack of neurodegeneration or slowed neurodegenerative progress. Additional samples may be obtained at subsequent time points to provide a third, fourth, fifth, sixth, seventh, eighth, ninth, tenth, eleventh, twelfth, thirteenth, fourteenth, fifteenth, or more time points.

Another aspect of the invention provides a method of diagnosing ND in a subject. In some aspects, the method includes obtaining a sample comprising platelets from the subject; adding a binding agent of TDP-43 protein to the sample; subjecting the sample to conditions allowing the formation of the binding complexes between the binding agent and the TDP-43 protein; measuring the amount of the binding complexes; and comparing the amount of TDP-43 protein with a control sample, such that an increased amount of the binding complexes in the sample in comparison to that in the control sample from a non-ND subject is indicative of presence of ND in the subject.

Yet another aspect of the invention provides a method of monitoring (ND) in a subject. In some aspects, the method includes obtaining a sample comprising platelets from the subject; adding a binding agent of TDP-43 protein to the sample; subjecting the sample to conditions allowing the formation of the binding complexes between the binding agent and the TDP-43 protein; measuring the amount of the binding complexes; and comparing that with a control sample, such that an increased amount of the binding complexes in the sample in comparison to that in the control sample from the subject at a previous time point is indicative of progression of ND in the subject.

An increase in the level or amount of TDP-43 protein compared to that of a control sample or standard is indicative of ND. Such increase may be an increase of 5-100%, or more. In some aspects, the increase is about 5, 10, 15, 20, 25, 30, 35, 40, 45, 50, 55, 60, 65, 70, 75, 80, 85, 90, 95, 100% or more. Preferably, the increase is about 10, 15, 20, 25, 30, 35, 40, 50, 55, 60, 65, 70% or more. More preferably, the increase is at least 15%.

A decrease in the level or amount of TDP-43 protein compared to that of a control sample or standard is indicative of ND improvement. Such improvement is an indication that the progression of neurodegeneration has slowed or ceased. A suitable decrease may be a decrease of 5-100%, or more. In some aspects, the decrease is about 5, 10, 15, 20, 25, 30, 35, 40, 45, 50, 55, 60, 65, 70, 75, 80, 85, 90, 95, 100% or more. Preferably, the decrease is about 10, 15, 20, 25, 30, 35, 40, 50, 55, 60, 65, 70% or more. More preferably, the decrease is at least 15%.

Still another aspect of the present invention provides a kit for evaluating ND in a subject, and the kit comprises at least one reagent for determining a level of TDP-43 protein in a biological sample comprising platelets.

Other aspects and iterations of the invention are described in more detail below.

### BRIEF DESCRIPTION OF THE FIGURES

The application file contains at least one photograph executed in color. Copies of this patent application publication with color photographs will be provided by the Office upon request and payment of the necessary fee. The following drawings form part of the present specification and are included to further demonstrate certain aspects of the present invention. The invention may be better understood by reference to one or more of these drawings in combination with the detailed description of specific embodiments presented herein.

FIG. 1A shows Western blotting of TDP-43 in the AD brain regions and is representative of three individual experiments.

FIG. 1B graphically illustrates the Western blot analysis for TDP-43 Protein levels in the Alzheimer's disease (AD) brain regions compared to control brain regions. The error bars represent the average of three subjects.

FIG. 2 shows TDP-43 protein levels in human platelet homogenate which were analyzed by Western blotting.

FIG. 3 shows TDP-43 aggregation in human AD brain regions including the frontal cortex (lane 1), Cerebellum (lane 2), and Hippocampus (lane 3). The protein aggregation was more pronounced in the frontal cortex and Hippocampus.

FIG. 4 shows antibody screening for one AD (FIG. 4A) and one ALS (FIG. 4B) patient's platelet homogenate. Six different anti-phospho TDP-43 and anti-TDP-43 antibodies with different titrations were used.

FIGS. 5A, 5B and 5C depict Western blots showing detection of phosphorylated TDP-43 protein using pS410/409 antibody. Detection was performed in triplicate (blot numbers 1, 2, and 3) and the triplicate procedure was repeated over three days (FIGS. 5A, 5B, and 5C, respectively). Lane numbers presented below each lane correspond to: #1, control sample; #2, AD sample; and #3, ALS sample. Arrows show the predominant phosphorylated TDP-43 protein (74 kDa) specific to a platelet from an AD subject.

FIG. 6 depicts a plot showing the levels of phosphorylated TDP-43 protein in control, ALS, and AD samples using the Luminex platform;

FIG. 7 depicts a virtual blot image of a capillary Western blot showing a phosphorylated TDP-43 protein band in AD samples (arrow) but not in control or ALS samples.

### DETAILED DESCRIPTION OF THE INVENTION

The present invention relates to the detection of TDP-43 protein, which aggregates in degenerating cells during neurodegenerative conditions or diseases. By analyzing levels of TDP-43 protein in platelets of a subject, it is possible to detect and monitor neurodegenerative conditions associated with TDP-43 aggregation. Further, by monitoring for changes in TDP-43 levels in platelets, the presence of neurodegenerative diseases or conditions can be predicted. Accordingly, the present invention provides compositions and methods useful in research, diagnostics, and therapeutics for conditions and diseases associated with neurodegeneration. The compositions and methods are directed to detecting TDP-43 proteins and peptides in platelets. TDP-43, as a platelet biomarker, makes a non-invasive, convenient and accurate diagnostic tool available in the process of diagnosis and treating of neurodegenerative diseases or conditions. Preferably, the neurodegenerative condition or disease is detected early.

Various aspects of the invention are described in further detail in the following subsections.

### I. Compositions

TDP-43 is a nucleic acid binding protein that forms intracellular aggregates in degenerating nerve cells. TDP-43 is encoded by the nucleic acid sequence of SEQ ID NO:1 and the amino acid sequence of SEQ ID NO:2. The terms “TDP-43” or “TDP-43 protein” are used herein to refer to all forms of TDP-43, which include full length TDP-43 protein, truncated TDP-43 protein, peptides of TDP-43 protein, fragments of TDP-43 protein, post-transcriptionally modified TDP-43 protein, phosphorylated TDP-43 protein, hyper phosphorylated TDP-43 protein, ubiquinated TDP-43 protein, and fragments or peptides of post-transcriptionally modified TDP-43 protein. Post transcriptional modifications include, without limitation, phosphorylation, hyper phosphorylation, ubiquitination, SUMOylation, glycosylation, lipidation, oxidation, methylation, cystinylation, sulphonation, acetylation, and modifications known in the art. Phosphorylated TDP-43 protein refers to TDP-43 protein or peptide with at least one phosphorylation site that has been phosphorylated. Hyper phosphorylated TDP-43 protein refers to TDP-43 protein or peptide that is fully saturated at the multiple phosphorylation sites.

In one aspect, the TDP-43 detected in a sample is not phosphorylated. In another aspect, the TDP-43 detected in a sample has at least one phosphorylation site that has been phosphorylated. In another embodiment, the TDP-43 detected in a sample has 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, or more phosphorylation sites that have been phosphorylated.

In one aspect, the TDP-43 detected in a sample is full length TDP-43. In another aspect, the TDP-43 detected in a sample is truncated TDP-43. In another aspect, the TDP-43 detected in a sample includes full length and truncated TDP-43. Truncated TDP-43 includes any amino acid fragments having 70 to 100% homology to SEQ ID NO: 2. Preferably, truncated TDP-43 has 70, 75, 80, 81, 82, 83, 84, 85, 86, 87, 88, 89, 90, 91, 92, 93, 94, 95, 96, 97, 98, 99, 100% homology to SEQ ID NO:2.

A binding agent may be used to detect TDP-43 protein in samples. Suitable binding agents include molecules capable of binding to TDP-43. Such binding agents include, without limitation, a nucleic acid, oligonucleic acid, amino acid, peptide, polypeptide, protein, lipid, metabolite, small molecule, antibody, fragment thereof, or combination thereof that is capable of binding to SEQ ID NO: 2 or a portion thereof. Preferably, the binding agent is an antibody or fragment thereof that is capable of recognizing TDP-43 proteins or fragments.

In one aspect, the binding agent may recognize at least one compound of the group consisting of TDP-43, phosphorylated TDP-43, hyper phosphorylated TDP-43, ubiquinated TDP-43, SUMOylated TDP-43, TDP-43 truncation, or TDP-43 fragments. In another aspect, the binding agent may recognize at least two compounds from the group consisting of TDP-43, phosphorylated TDP-43, hyper phosphorylated TDP-43, ubiquinated TDP-43, glycosylated TDP-43, TDP-43 truncation, or TDP-43 fragments. In another aspect, the binding agent may recognize at least three compounds from the group consisting of TDP-43, phosphorylated TDP-43, hyper phosphorylated TDP-43, ubiquinated TDP-43, glycosylated TDP-43, TDP-43 truncation, or TDP-43 fragments. In another aspect, the binding agent may recognize at least four compounds from the group consisting of TDP-43, phosphorylated TDP-43, hyper phosphorylated TDP-43, ubiquinated TDP-43, SUMOylated TDP-43, TDP-43 truncation, or TDP-43 fragments. In another aspect, the binding agent may recognize all forms of TDP-43 protein from the group consisting of TDP-43, phosphorylated TDP-43, hyper phosphorylated TDP-43, ubiquinated TDP-43, glycosylated TDP-43, TDP-43 truncation, or TDP-43 fragments. In an exemplary aspect, the binding agent may recognize one form of TDP-43 from the group consisting of TDP-43, phosphorylated TDP-43, hyper phosphorylated TDP-43, ubiquinated TDP-43, glycosylated TDP-43, TDP-43 truncation, or TDP-43 fragments and not substantially recognize the remaining forms of TDP-43. By way of example, without limitation, the binding agent may recognize hyper phosphorylated TDP-43 and not substantially recognize non-phosphorylated TDP-43, ubiquinated TDP-43, glycosylated TDP-43, TDP-43 truncation, or TDP-43 fragments.

A binding agent of the invention “recognizes” a compound if the IC₅₀ ratio is greater than about 20%. In one aspect, a binding agent of the invention has an IC₅₀ ratio of greater than about 20%, greater than about 23%, greater than about 25%, greater than about 27%, greater than about 30%, greater than about 33%, greater than about 35%, greater than about 37%, greater than about 40%, greater than about 43%, greater than about 45%, greater than about 47%, greater than about 50%, greater than about 53%, greater than about 55%, greater than about 57%, greater than about 60%, greater than about 63%, greater than about 65%, greater than about 67%, greater than about 70%, greater than about 73%, greater than about 75%, greater than about 77%, greater than about 80%, greater than about 83%, greater than about 85%, greater than about 87%, greater than about 90%, greater than about 93%, greater than about 95%, greater than about 97%, or greater than about 100% for TDP-43.

A binding agent of the invention “substantially does not recognize” a compound if the IC₅₀ ratio is less than about 15%. In one aspect, a binding agent of the invention has an IC₅₀ ratio of less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or less than about 1% for TDP-43.

In one aspect of the invention, the binding agent is an antibody. The antibodies of the invention may be murine antibodies, rabbit antibodies, human antibodies, humanized antibodies, chimeric antibodies, recombinant antibodies, or antibody fragments thereof without departing from the scope of the invention. In one aspect, the invention encompasses murine antibodies or fragments. Non-limiting examples of murine antibodies include mouse and rat antibodies. In another aspect, the invention encompasses human antibodies or fragments. In yet another aspect, the invention encompasses humanized antibodies or fragments. In still yet another aspect, the invention encompasses chimeric antibodies or fragments. In an alternative aspect, the invention encompasses recombinant antibodies or fragments. In another alternative, the invention encompasses antibody fragments. Non-limiting examples of such fragments include Fab fragments, Fab′ fragments, F(ab′)₂ fragments, single chain antigen binding fragments (scFv), disulfide stabilized Fv (dsFv) fragments, single domain antigen binding fragments, and other antibody fragments that maintain the binding specificity of the whole antibody but that are more cost-effective to produce, more easily used, or more sensitive than the whole antibody.

Antibodies may have lambda, kappa, or a recombinant light chain. Additionally, antibodies are typically, but not necessarily, IgG antibodies. In certain aspects, the IgG antibodies may include antibodies from the IgG1, IgG2, IgG3, and IgG4 human antibody classes. In other aspects, the IgG antibodies may include antibodies from the IgG1, IgG2a, IgG2b, and IgG3 mouse antibody classes.

Binding agents, including the antibodies, may be further associated with a detectable moiety, which may be a chromogenic moiety (e.g., indoxyl, ONP, PNP, TMB), a fluorogenic moiety (e.g., methylumbelliferyl, methylcoumarin) or a light emitting moiety (e.g., D-luciferin Firefly). Additional detectable tags for antibodies include a radioactive isotope or chelate thereof, dye stain, enzyme, metal, or any other tags can be used to provide enhanced detection. One or more of such detectable moieties enables quantitative evaluation of TDP-43 protein via suitable detecting equipment, e.g., a spectrophotometer, fluorometer or luminometer, or even a human eye to differentiate a sample having high expression of a protein biomarker from a sample having low or no expression of the biomarker. In addition, molecular weight markers can also be utilized to provide information about protein mass, especially if the protein goes through post-translational modification due to a disease condition, the molecular weight change is then a good indicator. Further, an anti-TDP-43 antibody may be further associated with one or more secondary antibodies, which are capable of binding to the anti-TDP-43 antibody and increase the assay sensitivity by amplifying the signal.

In certain aspects, the binding agent is an antibody. Suitable antibodies will discern between phosphorylated TDP-43, hyper phosphorylated TDP-43, and non-phosphorylated TDP-43. In some aspects, the antibody may bind to the epitope of SEQ ID NO: 3. In some aspects, the antibody may bind to an epitope having about 75%, 80%, 81%82%, 83%, 84%, 85%, 86%, 87%, 88%, 89%, 90%, 91%, 92%, 93%, 94%, 95%, 96%, 97%, 98%, 99%, or 100% sequence homology in comparison to SEQ ID NO: 3. In some aspects, the antibody may bind to an epitope having the serine in position six of SEQ ID NO: 3 phosphorylated. In some aspects, the antibody may bind to an epitope having the serine in position seven of SEQ ID NO: 3 phosphorylated. In some aspects, the antibody may bind to an epitope having the serine in position six and the serine in position seven of SEQ ID NO: 3 both phosphorylated.

### II. Methods

The present invention provides methods for detecting TDP-43 protein in platelets. The methods disclosed herein are less invasive and more sensitive than cerebrospinal fluid biomarker detection, brain imaging scans, and other methods employed. As non-limiting examples, methods of the invention may be useful for detecting TDP-43 protein in platelets; detecting ND; monitoring ND; detecting post-translationally modified TDP-43; or for other reasons that become apparent to a skilled artisan.

The methods of the present invention may be useful for detecting, diagnosing, or predicting neurodegenerative diseases or conditions. Such neurodegenerative diseases or conditions include those associated with damage or death of nerve tissue or nerve cells. Suitable neurodegenerative diseases or conditions include, without limitation, Alzheimer's disease, Amyotrophic lateral sclerosis, Cerebral palsy, Friedreich's ataxia, Huntington's disease, Lewy body disease, Multiple sclerosis, Parkinson's disease, Spinal muscular atrophy, and other diseases known in the art.

The present invention includes methods of detecting TDP-43 protein by contacting a TDP-43 binding agent to a sample containing platelets. Further, the presence of TDP-43 protein of the contacted sample may be detected relative to a control sample.

One aspect of the present invention provides methods of diagnosing ND, or monitoring the progress of ND over a period of time, by assessing the level of TDP-43 protein in a sample of a subject in comparison to the level of such in a control sample. In one aspect, the TDP-43 protein is post-transcriptionally modified. Therefore, another aspect of the present disclosure provides methods of diagnosing ND, or monitoring the progress of ND over a period of time, by assessing the level of post-transcriptionally modified TDP-43 protein in a sample of a subject in comparison to the level of such in a control sample. In another aspect, the TDP-43 protein is phosphorylated. Therefore, another aspect of the present disclosure provides methods of diagnosing ND, or monitoring the progress of ND over a period of time, by assessing the level of phosphorylated TDP-43 protein in a sample of a subject in comparison to the level of such in a control sample. In another aspect, the TDP-43 protein is hyper phosphorylated. Therefore, another aspect of the present disclosure provides methods of diagnosing ND, or monitoring the progress of ND over a period of time, by assessing the level of hyper phosphorylated TDP-43 protein in a sample of a subject in comparison to the level of such in a control sample.

In one aspect, the level of TDP-43 protein is determined by detecting the amount of binding complexes between TDP-43 protein and the TDP-43 binding agent. In another aspect, the level of post-transcriptionally modified TDP-43 protein is determined by detecting the amount of binding complexes between post-transcriptionally modified TDP-43 protein and the post-transcriptionally modified specific binding agent. In another aspect, the level of phosphorylated TDP-43 protein is determined by detecting the amount of binding complexes between phosphorylated TDP-43 protein and the phospho-specific binding agent. In another aspect, the level of hyper phosphorylated TDP-43 protein is determined by detecting the amount of binding complexes between hyper phosphorylated TDP-43 protein and the hyperphospho-specific binding agent.

Methods of detecting TDP-43 protein may comprise assessing the biomarker using immunoassays. Such immunoassays include those well known in the art. By way of example, without limitation, suitable immunoassays include Western blot, simple Western, capillary Western, ELISA (enzyme-linked immunosorbent assay), ELISpot (enzyme-linked immunosorbent spot) assay, radioimmunoassay (RIA), immunofluorescent assay, flow cytometry, immunoprecipitation, immunocytochemistry, immunohistochemistry, and immunoaffinity chromatography. Alternatively, TDP-43 levels may be detected and measured using high performance liquid chromatography (HPLC), mass spectrometry, protein microarray analysis, polyacrylamide gel electrophoresis (PAGE) analysis, isoelectric focusing, 2-D gel electrophoresis, stable isotope labeling with amino acids (SILAC), or any enzymatic assay.

In one aspect, the Western blot is used for assessing the level of TDP-43 protein in one or more platelet samples. Following separation of the biological sample with SDS-PAGE and subsequent transfer to a membrane, such as PVDF or nitrocellulose membrane, a TDP-43 specific antibody can be used to identify the TDP-43 protein in the samples. In another aspect, the Western blot is used for assessing the level of phosphorylated TDP-43 protein in one or more platelet samples. Following separation of the biological sample with SDS-PAGE and subsequent transfer to a membrane, one or more TDP-43 phosphorylation specific antibodies can be used to identify the TDP-43 protein in the samples.

A method of directly measuring protein phosphorylation may be carried out using radiolabeled ³²P-orthophosphate. After incubating a sample with radiolabeled ³²P-orthophosphate and obtaining the protein extracts from a sample, then SDS-PAGE or 2-dimensional gel electrophoresis may be used to separate proteins. The protein gel is eventually exposed to film to show the presence and the relative amount of phosphorylated proteins. As an alternative, phosphorylation-dependent antibodies may be produced in rabbits immunized with benzonyl phosphonate conjugated to keyhole limpet hemocyanin (KLH). This antibody broadly recognizes proteins containing phosphotyrosine. For better specificity, however, phosphorylation state-specific (phospho-specific) antibodies may be developed. In one aspect, the phospho-specific anti-TDP-43 antibody is developed by immunizing a rabbit with a synthetic phosphopeptide representing the amino acid sequence surrounding the phosphorylation site of the TDP-43 protein. The immune sera can be applied to a peptide affinity column to generate a highly specific immunoreagent.

In another aspect, ELISA is used for assessing the level of phosphorylated TDP-43 protein in one or more platelet samples. The assay utilizes a first antibody specific for TDP-43 protein, independent of the phosphorylation state. The TDP-43 protein in the platelet sample is bound to the first antibody. In one aspect, the first antibody is immobilized, for example, plate-coated. A second antibody specific for the phosphorylation site of TDP-43 is then added to specifically detect the phosphorylated TDP-43 protein fraction. In one aspect, the resulting signal from the assay is colorimetric. In another aspect, the resulting signal is fluorometric. The intensity of the resulting signal is directly proportional to the concentration of phosphorylated protein present in the original sample.

In yet another aspect, intracellular flow cytometry is used for assessing the level of TDP-43 protein in one or more platelet samples. In still another aspect, immunocytochemistry is used for assessing the level of TDP-43 protein in one or more platelet samples. In another aspect, immunohistochemistry is used for assessing the level of TDP-43 protein in one or more platelet samples.

Since ND is often associated with prolonged progression, there is a need for an accurate monitoring methodology, which will allow a physician to perform therapy adjustments according to the patients' condition and severity of the disorder and to determine the dose and duration of treatment. In addition, such accurate monitoring methodology of the level of a platelet protein marker enables evaluating the efficacy of different treatments to prevent or to ameliorate the ND. Therefore, according to another aspect of the present invention, a method of determining the changes of the level of TDP-43 protein in a sample obtained from a treated subject prior to, during or following the ND treatment, whereas the treatment may be either prophylactic, therapeutic or experimental, is contemplated. The level of TDP-43 protein monitored over a period of time serves as an indicator for the efficacy of treatment and thus can be used to adjust treatment. In another aspect of the present invention, a method of determining the changes of the phosphorylation profile of TDP-43 in a sample obtained from a treated subject prior to, during or following the ND treatment, whereas the treatment may be either prophylactic, therapeutic or experimental, is contemplated. The phosphorylation profile of TDP-43 protein monitored over a period of time serves as an indicator for the efficacy of treatment and thus can be used to adjust treatment.

Further, the method of diagnosing ND using TDP-43 protein levels in platelets involves setting standards of reference for normal or diseased subjects. Standards may be set for “normal” levels of TDP-43 by selecting non-ND subjects for a control group, characterized by gender, age, etc. Similarly, standards may be set for “progressive” ND, which may also be differentiated by different groups of people, characterized by the type and stage of ND, age, gender, etc. Therefore, diagnosing or monitoring ND may be performed by comparative analysis of the level of TDP-43 protein in the sample obtained from a test subject with reference to established standards.

Further, the method of diagnosing ND using TDP-43 protein levels in platelets involves setting standards of reference for normal or diseased subjects. Standards may be set for “normal” levels of TDP-43 by selecting non-ND subjects for a control group, characterized by gender, age, etc. Similarly, standards may be set for “progressive” ND, which may also be differentiated by different groups of people, characterized by the type and stage of ND, age, gender, etc. Therefore, diagnosing or monitoring ND may be performed by comparative analysis of the level of TDP-43 protein in the sample obtained from a test subject with reference to established standards.

In another aspect, the method of diagnosing ND using TDP-43 phosphorylation profiles in platelets involves setting standards of reference for normal or diseased subjects. Standards may be set for “normal” phosphorylation profiles of TDP-43 by selecting non-ND subjects for a control group, characterized by gender, age, etc. Similarly, standards may be set for “progressive” ND, which may also be differentiated by different groups of people, characterized by the type and stage of ND, age, gender, etc. Therefore, diagnosing or monitoring ND may be performed by comparative analysis of the phosphorylation profile of TDP-43 protein in the sample obtained from a test subject with reference to established standards.

In one aspect, diagnosis of an ND according to the present invention can be effected by comparing the level of TDP-43 in platelets of a tested subject to an established standard, or to that of one or more control subjects without ND. Whenever the level of TDP-43 of the test subject is determined as being higher, preferably statistically higher, than the standard, or the level of TDP-43 in the control(s), diagnosis is indicated as positive. On the other hand, whenever the level of TDP-43 in platelets in the test subject is determined as being substantially similar to the standard or the control(s), the diagnosis is indicated as negative.

In another aspect, diagnosis of an ND according to the present invention can be effected by comparing the TDP-43 phosphorylation profile in platelets of a tested subject to an established ND standard, or to that of one or more control subjects without ND or with a specific ND. Whenever the TDP-43 phosphorylation profile of the test subject is determined as being similar, preferably statistically similar, to the ND standard, or the TDP-43 phosphorylation profile of the ND positive control, diagnosis is indicated as positive. Alternatively, whenever the TDP-43 phosphorylation profile of the test subject is determined as being similar, preferably statistically similar to the negative standard, or the ND negative control, diagnosis is indicated as negative.

When the method provided herein is used for monitoring ND progression, the change of TDP-43 protein level, or phosphorylation profile, is at the center in determining whether the ND condition is worsening, improving or stable for a subject over a period of time. In this embodiment, samples are collected from the subject at predetermined points of time, and the level of the TDP-43 protein, or phosphorylation profile, at each time point is compared to that of previous time points.

It is envisioned that a wide variety of processing formats may be used in conjunction with the methods disclosed herein, including, without limitation, manual processing, partial automated-processing, semi-automated-processing, full automated-processing, high throughput processing, high content processing, and the like or any combination thereof.

### III. Kits

The present disclosure provides articles of manufacture and kits containing materials useful for detecting ND as described herein. The article of manufacture may include a container of a composition as described herein with a label. Suitable containers include, for example, bottles, vials, and test tubes. The containers may be formed from a variety of materials such as glass or plastic. The container holds a composition which is useful for detecting TDP-43 protein in samples containing platelets. The label on the container may indicate that the composition is useful for detecting ND and may also indicate directions for detection.

Preferably, the kit includes all the necessary materials, reagents and instructions for use and interpretation of results to facilitate the detection of TDP-43 protein in platelets. Accordingly, the kit comprises at least one reagent for determining a level of TDP-43 protein or post-translational modification thereof in a biological sample containing platelets. The kit further includes packaging material which identifies the reagent or reagents for use in detecting, diagnosing, or monitoring ND. In some aspects, the kit comprises at least one TDP-43 specific antibody reagent. The antibody reagent may be provided as a working solution, or as a freeze-dried powder. In one aspect, the kit comprises at least one TDP-43 phospho-specific antibody for forming binding complex specific to phosphorylated TDP-43 protein. The kit may further comprise a set of buffer solutions, a leaflet of instructions for use and a packaging material, which identifies the kit for use in diagnosing ND. Preferably, the TDP-43 specific antibody or the TDP-43 phospho-specific antibody is coupled to biotin or an enzyme, such as, but not limited to, horseradish peroxidase, alkaline phosphatase or β galactosidase. Alternatively, the reagent includes an antibody binding protein that is coupled to an enzyme, such as, but not limited to, horseradish peroxidase, alkaline phosphatase or β galactosidase. In another aspect, the kit includes at least one substrate of the enzyme. Preferably, the substrate includes a detectable moiety that is chromogenic, fluorogenic or light emitting. For example, tetramethylbenzidine chromogenic substrate for horseradish peroxidase, or methylumbelliferyl phosphate fluorogenic substrate for alkaline phosphatase.

### DEFINITIONS

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

The term “antibody” is used herein in the broadest sense and refers generally to a molecule that contains at least one antigen binding site that immunospecifically binds to a particular antigen target of interest. Antibody, thus, includes, but is not limited to, polyclonal antibodies, monoclonal antibodies, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term, thus, includes full length antibodies and/or their variants as well as immunologically active fragments thereof, thus encompassing, antibody fragments capable of binding to a biological molecule (such as an antigen or receptor) or portions thereof, including but not limited to Fab, Fab′, F(ab′)2, facb, pFc′, Fd, Fv or scFv (See, e.g., CURRENT PROTOCOLS IN IMMUNOLOGY, (Colligan et al., eds., John Wiley & Sons, Inc., NY, 1994-2001).

The sample for the present invention is preferably a biological sample from a subject. The terms “sample” or “biological sample” are used in its broadest sense. Depending upon the aspect of the invention, for example, a sample may comprise a bodily fluid including whole blood, serum, plasma, an extract from the blood, such as, platelet rich plasma (PRP), blood cells, lyphocytes, red blood cells, leukocytes, isolated platelets, and combinations thereof. Preferably, the sample is platelets, which may be a dry pellet, or suspended as platelet homogenate. The sample may be analyzed directly or alternatively it can be processed prior to analysis. Processing of the sample may include adding an anticoagulant (e.g., EDTA, heparin and acid-citrate-dextrose (ACD)), protease inhibitor, phosphatase inhibitor, or preservative. The sample may be analyzed whole, centrifuged or fractionated. The sample may be analyzed fresh, refrigerated or frozen.

The term “subject” is used in its broadest sense. In a preferred embodiment, the subject is a mammal. Preferably, a subject includes any human or non-human mammal, including for example: a primate, capable of developing ND or related dementia. A human subject may be one that is suspected of having ND but yet to be diagnosed, or one that has ND and is under a treatment which process can be monitored using the method provided herein. The method of detecting the level of TDP-43 protein in platelets may comprise extracting platelets from whole blood. Typically, a sufficient amount of blood (5-10 ml, for example) is centrifuged to separate the platelet-rich serum and the red cell-serum. The Platelet-rich serum is collected and further centrifuged to separate supernatant and platelets in the pellet at the bottom. The platelets pellet may be further washed in proper buffer, and then centrifuged again to obtain the platelet pellet. Once the trace amount of supernatant is aspirated from the pellet, the platelets may be suspended again to obtain the platelet homogenate for subsequent assay. Alternatively, gel filtration may be used to separate platelet protein from other proteins in a blood sample. For example, SDS polyacrylamide gradient gel, can be used to effectively separate platelets from plasma proteins using relatively small samples of blood which are not amenable to repeated centrifugation. Gradient gel can also resolve phosphorylated and dephosphorylated platelet proteins.

### Examples

The following examples are simply intended to further illustrate and explain the present invention. The invention, therefore, should not be limited to any of the details in these examples.

### Example 1

### Platelet TDP-43 Associates with Alzheimer's Disease (AD)

The brain samples from three AD patients, and three age-matched control subjects were obtained from the University of Kansas Alzheimer's disease Center (KUADC) Neuropathology Core Laboratories. The brain tissue samples consisted of frontal cortex (FCtx), cerebellum (Crblm), and hippocampus (Hipcmp), which brain regions are affected by Alzheimer's disease. The whole blood samples for both the AD and non-AD subjects were obtained from the KUADC Bio-specimen Unit.

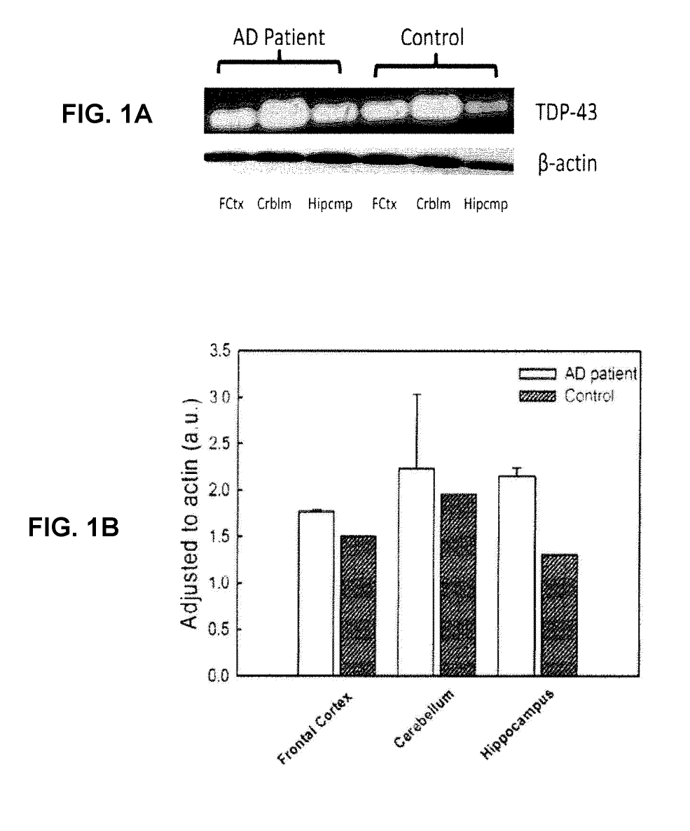

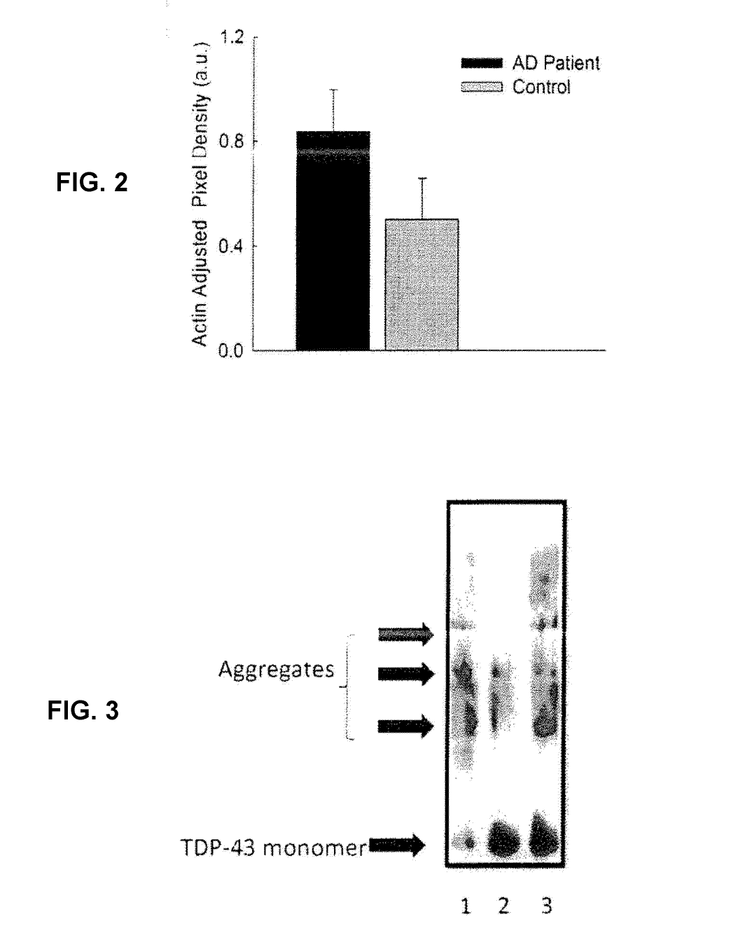

Western blot was used for detecting the TDP-43 protein in affected brain regions from AD patients. A TDP-43 specific antibody was produced and used in assaying each of the three AD brain samples. Compared to the non-AD control samples, AD brain regions of all three AD patients showed noticeable difference in TDP-43 protein levels, especially in hippocampal region, which presents the highest elevation of TDP-43 in AD patients (FIG. 1A). FIG. 1B further illustrates the quantification of TDP-43 levels in AD brain regions compared to that in brain regions of controls using Western blot analysis. The increase of the TDP-43 level of the three AD subjects was about 20% in frontal cortex, about 15% in cerebellum, and about 60% in hippocampus when comparing the AD versus non-AD (FIG. 1B). Therefore, increased TDP-43 protein levels in AD brain samples were observed as compared to age-matched control subjects. The increase in frontal cortex and hippocampus but not cerebellum is consistent with sites of highest AD pathology.

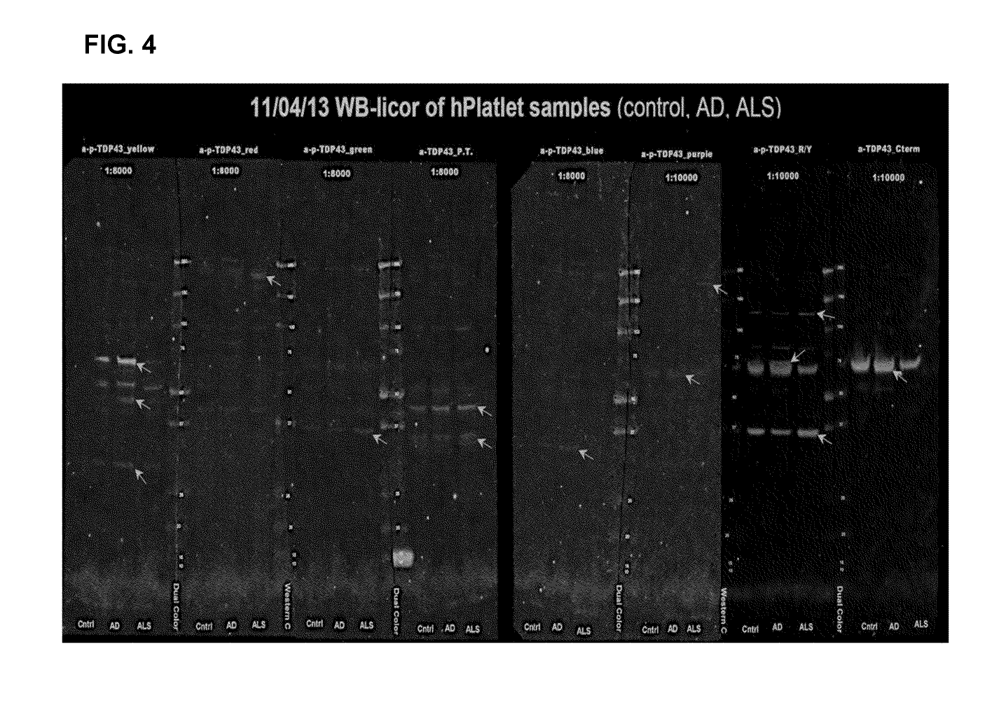

Subsequently, platelet proteins in platelet homogenate were immunoprobed with anti-TDP-43 antibody after resolving the platelet proteins in a 4-20% gradient gel. It was observed, using Western blotting analysis, that the TDP-43 protein levels increased about 40% in platelets from AD patients compared to controls (FIG. 2). These preliminary data suggest that the platelet TDP-43 protein levels mirror the TDP-43 protein alterations in the brain of a disorder, which could further be associated with hyper phosphorylated TDP-43. The phosphorylation status of TDP-43 in AD platelets, as well as AD brain samples, was being confirmed under immunoblotting assays using a phospho-specific anti-TDP-43 antibody.

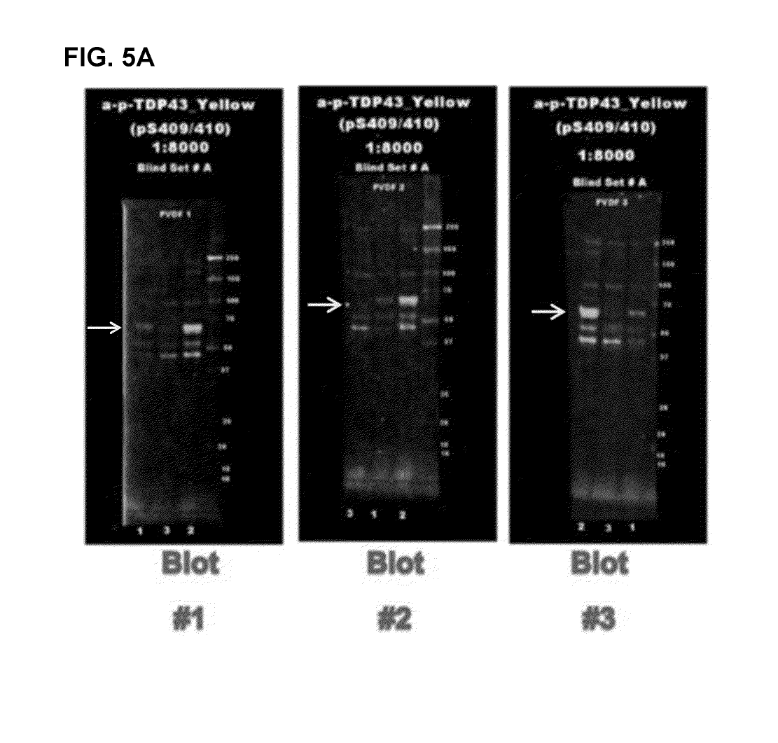

The electrophoretic mobility of the TDP-43 under the non-reducing conditions was tested as well. Major protein mobility retardation on the native gel was observed, indicating the formation of aggregated TDP-43 (FIG. 3). Additionally, platelets obtained from three (n=3) AD patients and three (n=3) age-matched control subjects were also analyzed for electrophoretic mobility of the TDP-43 under the non-reducing conditions.

### Example 2

### Platelet TDP-43 Phosphorylation Profile Indicative of Neurodegeneration

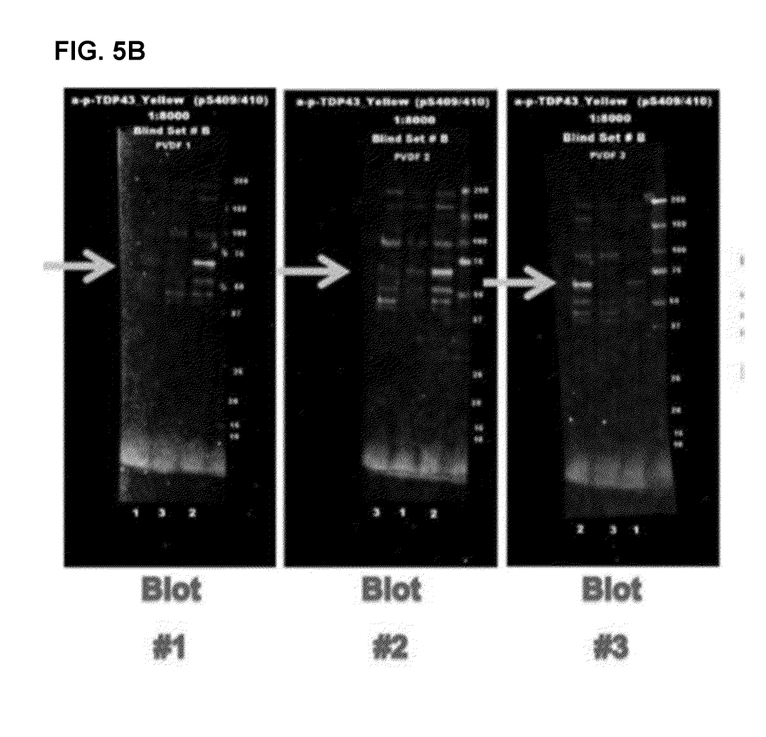

Platelet samples from one AD patient and one ALS patient were analyzed for TDP-43 protein levels. Western blot was used for detecting the TDP-43 protein in the platelet samples. Six different anti-phospho TDP-43 and anti-TDP-43 antibodies, at different titrations, were used for the analysis. The resulting profile for the AD sample (FIG. 4A) showed a noticeable difference in TDP-43 phosphorylation compared to the ALS sample (FIG. 4B). The detected phosphorylation statuses, recognized by the antibodies, are summarized in Table 1. The data support different profiles of phosphorylated TDP-43 in AD and ALS platelets.

TABLE 1
Phosphoyrlation profile of TDP-43 in AD and ALS.

| | Phosphorylation status | | | | | |
|---|---|---|---|---|---|---|
| Sample | pS | pS | pS | pS | pS | pS |
| Platelets | 409/410 | 409/410-1 | 409 | 403/404 | 410 | 409/410-12 |
| ALS | ~45 KDa | — | ~30 KDa | 28 KDa | ~35 KDa | ~130, 72, |
| | | | | and lower | | 35 KDa |
| AD | — | — | 35, 30, 22 | ~250, 53, | ~27 KDa | ~60-65 |
| | | | KDa | 10 KDa | | KDa |

### Example 3

### Detecting ND in a Subject Using Platelet TDP-43 Protein

Collect 5 ml, or more, of blood sample from a patient suspected of having a neurodegenerative disease. Store the whole blood in a container suitable for centrifugation, and mix gently during and after blood collection by slowly inverting the container. Spin at 200 g for 20 min at room temperature to prepare platelet-rich plasma (PRP) by centrifugation. The centrifugation conditions may vary widely (for example, from 800 g for 5 min to 100 g for 20 min) for providing consistently good separations. The top layer, which is PRP, is to be collected after centrifugation. A volume of 4-4.5 ml blood usually results in an average of 1-3×10⁸ platelets, which can vary depending on the individual the blood is drawn from. The PRP is suitable for the subsequent analysis, with further platelet isolation being optional. The PRP or isolated platelet is then to be incubated with anti-TDP-43 antibody to enable the binding of the antibody to the platelet TDP-43 proteins. The antibody/antigen complex will then be pulled out of the sample, for example, using protein A/G-coupled agarose beads. Alternatively, the isolated antibody/antigen complex may be separated by SDS-PAGE for western blot analysis-based quantification. The reading of the platelet TDP-43 protein levels is to utilize a pre-prepared standard curve showing the level of TDP-43 antibody/antigen complex level in relation to the volume of the whole blood, PRP or total platelet protein preparation. The standard curve is made using blood sample(s) collected from people without any diagnosis or clinical symptoms of neurodegenerative diseases. If the reading of the TDP-43 antibody/antigen complex level in the patient subjected to the test is above the curve, this is an indication that the patient has TDP-43 related neurodegenerative disease.

### Example 4

### Monitoring ND Using Platelet TDP-43 Protein Levels Detected During and after Treatment

Prior to a treatment, collect 5 ml, or more, of blood sample from the patient diagnosed with a neurodegenerative disease. This initial sample (Sample 0) is to be processed, analyzed and quantified for the platelet TDP-43 protein content using the procedures and methods provided above. The level of the platelet TDP-43 in Sample 0 is to be used for establishing a baseline level for evaluating treatment effect and disease progression during and after the treatment has been given to the patient.

During the treatment, blood samples from the same patient are to be collected periodically. Depending on the length of the treatment plan, the blood sample is to be collected, for example, every month for a three-month plan, or every other month for a six-month plan, or every three months for a year plan, or every four months for a two-year plan. Alternatively, the length of the treatment is to be determined by the change in the level of the platelet TDP-43 of the patient. For example, when the level of the platelet TDP-43 protein in the patient is reduced to a desired low level, or reaches to nearly zero, the patient may receive an additional cycle (i.e. one more month if the blood is collected once a month, or two more months if the blood is collected every other month, so on so forth) of treatment before treatment ceases. The monitoring of the disease after treatment continues by collecting and analyzing blood samples.

In this particular example, the blood sample is to be collected every month during the treatment until the level of platelet TDP-43 protein is close to the level of a non-ND standard control in two consecutive blood tests. Sample 1 is to be collected at one month after the treatment starts. This blood sample is to be analyzed, and the platelet TDP-43 protein level is to be measured and compared to the baseline level. The subsequent blood tests are to be carried out the same way. The platelet TDP-43 protein level from each blood test is to be plotted with previous numbers. If the level of the platelet TDP-43 protein in this patient is trending downward in comparison to the baseline and other previous measurements, it indicates that the patient is responding to the treatment, and the same treatment should continue. If the level of the platelet TDP-43 protein in this patient is trending upward, it indicates that the patient is not responding to the treatment and the disease may be still progressing. If the level of the platelet TDP-43 protein in this patient reaches a plateau during the treatment, it may indicate that the disease is stabilized under the treatment, and that the treatment may need to be enhanced, or in some cases, stay unchanged.

In the event that the treatment stops, either because the disease condition is relieved or because it is stabilized, the blood samples are still to be collected and analyzed to show the trend of the level change in the platelet TDP-43 protein in this patient. If the number starts to trend upward after the treatment stops, the patient and doctor may need to consider resuming the treatment. Otherwise, the patient will be under constant monitoring for the state of the neurodegenerative disease by checking the level of platelet TDP-43 protein periodically.

### Example 5

### Analysis of Phosphorylated TDP-43 Protein Using Immunoblotting

Platelet samples from an AD patient and an ALS patient were analyzed for TDP-43 protein levels. Western blot was used for detecting the TDP-43 protein in the platelet samples. The ps409/410 anti-phosphor TDP-43 antibody was used for the analysis at different titrations. The pS409/410 anti-phospho TDP-43 IgG1 antibody was raised in a mouse against the CMDSKS(p)S(p)GWGM phosphoserine 409/410 of TDP-43 (SEQ ID NO: 3). This antibody specifically recognized predominantly phosphorylated human TDP-43 protein species in the platelet homogenates obtained from Alzheimer's diseases (AD) patients.

The Alzheimer's (AD) patient and accompanying control platelet samples were obtained from the University of Kansas Medical Center bio-depository center, thawed, and adequate phosphatase and protease inhibitors were added before aliquoting and storing at −80° C. until use. The amyotrophic lateral sclerosis (ALS) patient's and accompanying control blood samples were obtained from the University of Kansas Medical Center ALS clinic. Platelets were isolated by two step low speed centrifugation and adequate phosphatase and protease inhibitors were added, aliquoted, and stored in −80° C. until use.

Platelet homogenate proteins (10 μg) were loaded onto 4-20% gradient gel (Mini-PROTEAN® TGX™ pre-cast gels BioRad) under reducing conditions. The proteins were separated by standard electrophoresis at 100 Volt for 90 minutes. The proteins were then transferred onto PVDF membranes by a standard electrotransfer method at 60 Volt for 90 minutes. The PVDF membrane was briefly washed with mega pure water and blocked in SEA Block blocking buffer (ThermoScientific) (1:1 diluted in 1×TBS buffer) for 1 hour at room temperature with agitation, then incubated in a primary antibody solution (anti-phospho TDP-43 mouse antibody (S409-410)(CosmoBio-USA) diluted 1:8000 dilution (this dilution ratio is optimized) in the SEAblock blocking buffer diluted in 1:1 ratio in TBST overnight at 4° C. with agitation. Next day, the PVDF membrane was washed 6 times for 5 minutes in TBST (TrisBufferSaline-Tween20). Anti-mouse (IRDye®RD) secondary antibodies were prepared at 1:10,000 dilution in seablock blocking buffer diluted in 1:1 ratio in TBST. The PVDF membrane was incubated for 2 hours at room temperature with agitation in a dark-colored incubation tray. The PVDF membrane was then washed 6 times for 5 minutes in TBST and one time for 5 minutes in TBS. The membrane was analyzed by the Odyssey Infrared Imaging System (LI-COR Biosciences). This procedure was performed in triplicate, in blinded fashion, and the triplicate procedure was repeated over three consecutive days.

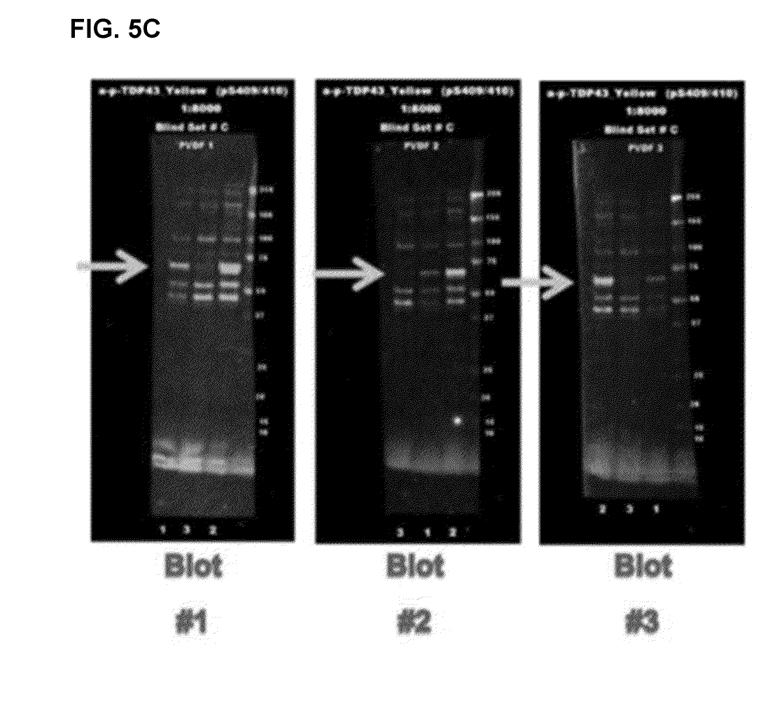

The resulting Western blotting profiles from each triplicate procedure consistently showed a predominant phosphorylated TDP-43 protein in the AD sample compared to the ALS and control samples (FIG. 5). The data confirm the different profiles of phosphorylated TDP-43 in AD and ALS platelets shown in Example 2, and support the ability of an antibody capable of recognizing the CMDSKS(p)S(p)GWGM phosphoserine 409/410 of TDP-43 to detect phosphorylated TDP-43 using Western blotting techniques, and diagnosing AD using a platelet homogenate sample from a subject.

### Example 6

### Analysis of Phosphorylated TDP-43 Protein Using Flow Cytometry

This platform assesses the presence of phosphorylated TDP-43 in an intact platelet population prepared from CONTROL subjects, AD, and ALS patient's blood. An initial analysis may be performed using anti-phospho TDP-43 (pS409/410) and anti phospho TDP-43 (pS409) antibodies using fixed platelets obtained from control, AD, and ALS patients to determine the best method for successful phosphor-epitope detection. The flow cytometry protocol comprises two steps:

Step 1: Sample Preparation.

Whole blood was drawn into an ACD-containing vacutainer tube and mixed gently 3-5 times. The sample was subjected to low speed centrifugation at 200 g for 15-20 minutes at room temperature. Platelet rich plasma (PRP) was carefully removed leaving about 0.2 ml so that buffy coat was not disturbed. PRP aliquot (depending on PRP volume ranging between 0.25 ml-2 ml) was transferred to a polypropylene 15 mL conical tube or a polypropylene Eppendorf tube. To each tube, Fixation Buffer 1 was added and mixed by pipetting or gently vortexing. The volume of Fixation Buffer added may be ⅕ the PRP volume. The samples were incubated for 15 minutes at room temperature. During the fixation, PRP samples were divided into 25-50 μL aliquots in polypropylene Eppendorf tubes. Following the 15 minute fixation, the fixed PRP samples immediately were transferred to a −80° C. freezer for storage until use.

Step 2: Platelet Analysis.

Each PRP sample was analyzed by flow cytometry to evaluate the following characteristics:
Light scatter characteristics (approximate cell size)
Staining of the pan platelet surface markers CD61 and CD41a
Staining of the platelet activation marker CD62P
Platelet count
Platelet viability

### Example 7

### Analysis of Phosphorylated TDP-43 Protein Using a Bead-Based Assay

This platform assesses the presence of phosphorylated TDP-43 using bead-based fluorescence detection which operates on the principles of flow cytometry. This system combines flow cytometry with differentially dyed microspheres to allow simultaneous analysis of up to 100 different targets in a single sample providing clinical laboratories with the ability to do very high levels of multiplex detection of either nucleic acid or protein targets. Each individual color/region is then coupled with reagents specific to the assays of interest (e.g. anti-phospho TDP-43 antibody). The microspheres are magnetically charged allowing for quick and reliable processing. These beads can be mixed into a single well (sample) for multiplexing. Up to 50 assays can be multiplexed in a single well. The microsphere internal dyes are excited by a laser, marking the microsphere set. A second laser excites the fluorescent dye on the reporter molecule. Finally, high-speed digital-signal processors identify each individual microsphere and quantify the result of its assay, based on fluorescent reporter signals.

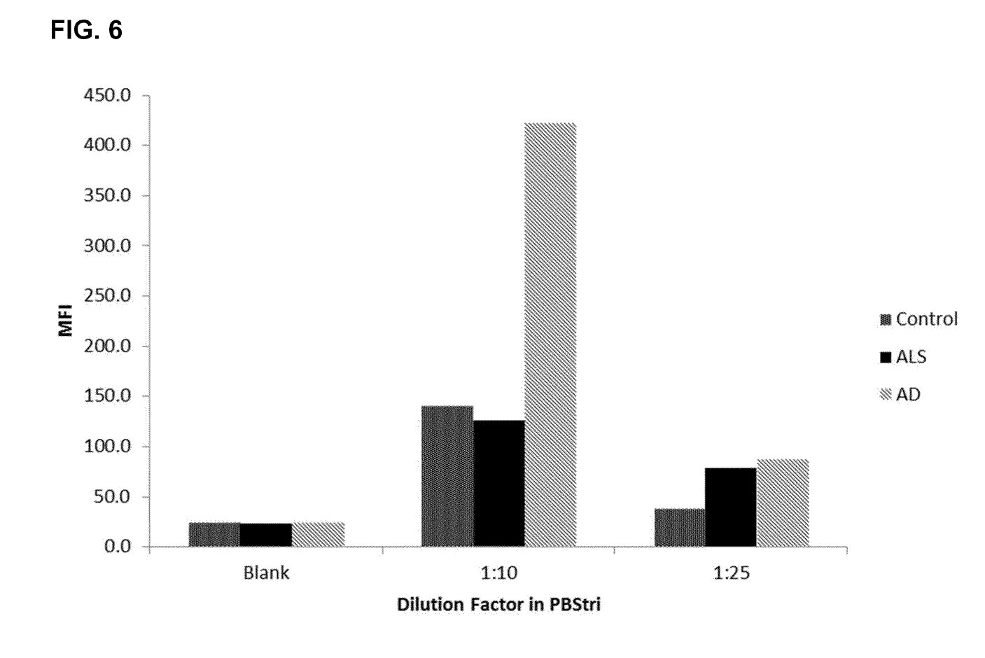

Phosphorylated TDP-43 was detected and measured using the Luminex Platform bead-based platform. Antibody and assay controls were used to verify assay performance. Control, ALS, and AD samples were used to assess the specificity of the assay (FIG. 6). The results show that beads coupled with anti-phospho TDP-43 (S410/409) specifically recognized platelets from AD samples that have relatively high levels of phosphorylated TDP-43. The levels of phosphorylated TDP-43 were approximately 3 times higher in AD samples when compared to ALS and control samples.

### Example 8

### Analysis of Phosphorylated TDP-43 Protein Using a Capillary Column Western Assay

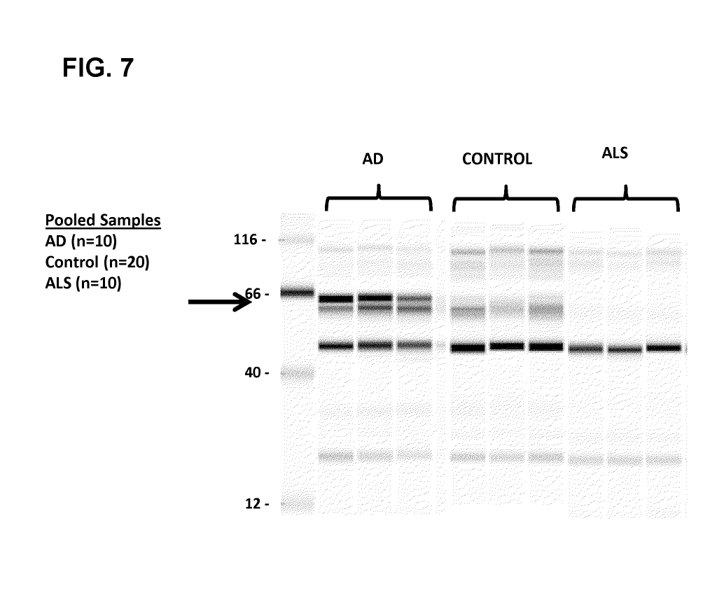

This platform performs free solution Westerns in a capillary column and detects protein zones in the capillary column. In this assay, the ProteinSimple Wes simple Western platform was used to perform the capillary column western assay. Simple Westerns can be done in one of two ways—separation of proteins by size or by charge. Sample preparation and analysis was performed following the manufacturer's protocol. In short, 5 μg platelet homogenate protein was loaded per micro capillary column for analysis. Chromatography run time was 50 minutes at 250V. Three individual replicates from pooled samples of control, ALS, and AD subjects were analyzed. The results demonstrate that AD platelets showed a unique phosphorylated TDP-43 protein species as compared to control and ALS platelets (FIG. 7).

The invention illustratively disclosed herein suitably may be practiced in the absence of any element, which is not specifically disclosed herein. It is apparent to those skilled in the art, however, that many changes, variations, modifications, other uses, and applications to the method are possible, and also changes, variations, modifications, other uses, and applications which do not depart from the spirit and scope of the invention are deemed to be covered by the invention, which is limited only by the claims which follow.

What is claimed is:

1. A method for detecting neurodegenerative disease, comprising:
a. obtaining a sample comprising platelets from a subject;
b. adding a TDP-43 binding agent to the sample;
c. measuring the amount of the binding complexes; and,
d. comparing the sample with a control sample, wherein an increased amount of the binding complexes in the sample in comparison to the amount of the binding complexes in the standard is indicative of ND in the subject.

2. The method of claim 1, wherein the sample contains platelets selected from the group consisting of isolated platelets, platelet rich plasma, plasma, blood, or mixtures thereof.

3. The method of claim 1, wherein the TDP-43 binding agent binds to TDP-43 protein selected from the group consisting of phosphorylated TDP-43, hyper phosphorylated TDP-43, ubiquinated TDP-43, truncated TDP-43, TDP-43, and combinations thereof.

4. The method of claim 1, wherein the TDP-43 binding agent is selected from the group consisting of TDP-43 specific antibody, TDP-43 phosphor-specific antibody, and combinations thereof.

5. The method of claim 1, wherein measuring the amount of the binding complexes is effected via an immunoassay.

6. The method of claim 6, wherein said immunoassay is selected from the group consisting of enzyme-linked immunosorbent assay (ELISA), a radioimmunoassay, an immunofluorescence assay, and a light emission immunoassay.

7. The method of claim 1, wherein the binding agent comprises at least one label for enhanced detection selected from the group consisting of a chromogenic moiety, a fluorogenic moiety, a light emitting moiety, a radioactive isotope or chelate thereof, a dye stain, an enzyme, and a metal.

8. The method of claim 1, wherein the detected neurodegenerative disease is selected from the group consisting of Alzheimer's disease, Parkinson's disease, corticobasal degeneration, Lewy-body dementia, frontotemporal lobar degeneration, amyotrophic lateral sclerosis, nerve degeneration, and combinations thereof.

9. A method of detecting neurodegenerative disease (ND), the method comprising:
a. obtaining a first sample comprising platelets from a subject;
b. adding a binding agent of TDP-43 protein to the first sample;
c. subjecting the first sample to conditions allowing the formation of the binding complexes between the binding agent and the TDP-43 protein;
d. measuring the amount of the binding complexes in the first sample;
e. obtaining a second sample comprising platelets from the subject;
f. adding a binding agent of TDP-43 protein to the second sample;
g. subjecting the second sample to conditions allowing the formation of the binding complexes between the binding agent and the TDP-43 protein;
h. measuring the amount of the binding complexes in the second sample; and
i. comparing the amount of the binding complexes in the first sample to the amount of the binding complexes in the second sample, wherein an increased amount of the binding complexes in the second sample in comparison to the amount of the binding complexes in the first sample is indicative of progression of ND in the subject, and wherein a decreased amount of the binding complexes in the second sample in comparison to the amount of the binding complexes in the first sample is indicative of improvement of ND in the subject.

10. The method of claim 9 further comprising obtaining subsequent samples from the subject.

11. The method of claim 9, wherein the sample contains platelets selected from the group consisting of isolated platelets, platelet rich plasma, plasma, blood, or mixtures thereof.

12. The method of claim 9, wherein the TDP-43 binding agent binds to TDP-43 protein selected from the group consisting of phosphorylated TDP-43, hyper phosphorylated TDP-43, ubiquinated TDP-43, truncated TDP-43, TDP-43, and combinations thereof.

13. The method of claim 9, wherein the TDP-43 binding agent is selected from the group consisting of TDP-43 specific antibody, TDP-43 phosphor-specific antibody, and combinations thereof.

14. The method of claim 9, wherein measuring the amount of the binding complexes is effected via an immunoassay.

15. The method of claim 14, wherein said immunoassay is selected from the group consisting of enzyme-linked immunosorbent assay (ELISA), a radioimmunoassay, an immunofluorescence assay, and a light emission immunoassay.

16. The method of claim 9, wherein the binding agent comprises at least one label for enhanced detection selected from the group consisting of a chromogenic moiety, a fluorogenic moiety, a light emitting moiety, a radioactive isotope or chelate thereof, a dye stain, an enzyme, and a metal.

17. The method of claim 9, wherein the detected neurodegenerative disease is selected from the group consisting of Alzheimer's disease, Parkinson's disease, corticobasal degeneration, Lewy-body dementia, frontotemporal lobar degeneration, amyotrophic lateral sclerosis, nerve degeneration, and combinations thereof.

18. A method of diagnosing neurodegenerative disease (ND), the method comprising:
a. obtaining a sample comprising platelets from a subject;
b. adding a binding agent of phosphorylated TDP-43 protein to the sample;
c. subjecting the sample to conditions allowing the formation of the binding complexes between the binding agent and the phosphorylated TDP-43 protein; and
d. measuring the amount of the binding complexes and comparing that with a control sample, wherein an increased amount of the binding complexes in the sample in comparison to that in the control sample from a non-ND subject is indicative of presence of ND in the subject.

19. A method of monitoring neurodegenerative disease (ND), the method comprising:
a. obtaining a sample comprising platelets from a subject;
b. adding a binding agent of phosphorylated TDP-43 protein to the sample;
c. subjecting the sample to conditions allowing the formation of the binding complexes between the binding agent and the phosphorylated TDP-43 protein; and
d. measuring the amount of the binding complexes and comparing that with a control sample, wherein an increased amount of the binding complexes in the sample in comparison to that in the control sample from the subject at a previous time point is indicative of progression of ND in the subject.

20. A kit for evaluating neurodegenerative disease (ND) in a subject comprising at least one reagent for determining a level of TDP-43 protein in a biological sample comprising platelets.

21. The kit of claim 20, wherein the reagent includes a TDP-43 protein specific antibody.

22. The kit of claim 20, wherein the TDP-43 protein is recognizable by a phosphor-specific anti-TDP-43 antibody.

23. The kit of claim 20, wherein the antibody includes at least one label for enhanced detection selected from the group consisting of a chromogenic moiety, a fluorogenic moiety, a light emitting moiety, a radioactive isotope or chelate thereof, a dye stain, an enzyme, and a metal.
